(19) United States
(12) Patent Application Publication
(10) Pub. No.: US 2025/0366716 A1
(43) Pub. Date: Dec. 4, 2025

(54) PLASMONIC NANOBUBBLE ENDOSCOPE SYSTEM FOR IN VIVO THERANOSTICS OF CANCER CELLS IN TISSUE

(71) Applicant: Scorpido Photonics Inc., San Pedro, CA (US)

(72) Inventor: Dmitri Lapotko, San Pedro, CA (US)

(21) Appl. No.: 19/233,803

(22) Filed: Jun. 10, 2025

Related U.S. Application Data
(63) Continuation-in-part of application No. PCT/US23/83308, filed on Dec. 11, 2023.
(60) Provisional application No. 63/432,010, filed on Dec. 12, 2022.

(51) Int. Cl. A61B 5/00; A61B 18/20; A61B 18/24

(52) U.S. Cl. CPC A61B 5/0062; A61B 5/0082; A61B 5/4836; A61B 5/4848; A61B 5/7271; A61B 18/24; A61B 2018/2035; A61B 2560/0462; A61B 2562/0233

(57) ABSTRACT

The invention relates to minimally invasive methods, devices and systems for diagnosis and/or connected diagnosis and treatment (theranostics) of disease (for example, cancer) at the cellular level in vivo through the generation and detection of disease-specific plasmonic nanobubbles (“PNBs”). PNBs are on-demand laser pulse-activated non-stationary vapor nanobubbles. A system for diagnosing and treating the disease in a patient comprises a laser module connected to a flexible fiber optical PNB probe, which optically generates and detects PNBs. A method for diagnosing the disease comprises (a) administering nanoparticles of small size, below 100 nm, for example, titanium nitride nanoparticles, or their disease-specific conjugates to a patient; (b) navigating a fiber optical probe in a patient to a target tissue; (c) generating PNBs in vivo with an infrared laser pulse of the duration longer than 200 ps, delivered through the fiber optical probe; (d) detecting PNBs optically in vivo with the said fiber optical probe through the optical backscattering by PNBs; and (e) diagnosing the disease through analysis of the detected optical signals in response to one or several laser pulses. The method further comprises treating the disease, based on the diagnostic step, with PNBs or other means.

### RELATED APPLICATIONS

The present disclosure is a continuation-in-part of International Patent Application No. PCT/US2023/083308, filing date 11 Dec. 2023, which claims the benefit of U.S. Provisional Patent Application No. 63/432,010, filing date 12 Dec. 2022. The above related applications are hereby incorporated by reference herein in their entirety.

### BACKGROUND OF THE DISCLOSURE

The disclosure relates to methods, device and system for diagnosis and/or connected diagnosis and treatment (“theranostics”) of disease (for example, cancer) at the cellular level in vivo through plasmonic nanobubbles (“PNBs”), as they were described in the parent PCT application and support parent claims.

The disclosure provides an additional design, data and examples in support of the original claims of the parent application (also included below), and reports a novel PNB method and device with the improved clinical usability, efficacy, safety, costs and manufacturability of the PNB device.

The disclosure describes a novel medical diagnostic and/or theranostic device and method through all-optical and remote generation and detection of PNBs in hard-to-reach and damage-sensitive locations and targets, including biological tissues and organs, through the combination of six components: (1) low-cost, thermally-resistant and biocompatible titanium nitride (TiN) nanoparticles with infrared optical absorbance, such nanoparticles efficiently convert infrared laser pulses into PNBs, (2) infrared laser pulses of relatively long duration, above 200 ps, so such pulses are biocompatible, available from compact low-cost lasers and can be delivered via standard solid optical fibers, (3) standard optical fiber with a solid core for the delivery of infrared laser pulses to the target tissue or organ with TiN nanoparticles, and (4) all-optical generation and detection of PNBs with the one device, through specific pattern and design of the optical fibers in the optical fiber bundle (referred to as “a PNB probe”), (5) a remote generation and detection of PNBs, (6) generating and detecting PNBs at one position of the PNB probe and (7) minimizing the invasiveness of the PNB generation and detection in vivo through the reduced size and improved flexibility of the fiber optical flexible PNB probe.

The novelty of the disclosure is in the above-described combination of long infrared laser pulse, small TiN nanoparticles and fiber optical PNB probe for all-optical generation and detection of PNBs, as detailed below.

Plasmonic nanobubbles are on-demand cell-level photomechanical nanoevents, non-stationary vapor nanobubbles generated around laser pulse-heated plasmonic nanoparticle (NP) clusters self-assembled by cancer cells [1-6]. In our preclinical in vivo studies, PNBs improved cancer diagnostics, therapy and surgery with instant direct detection and selective destruction of microscopic residual tumors, otherwise undetectable, therapy-resistant and unresectable [1,6-8].

In challenging cancers where local recurrence hinders the outcome and quality of life [9-25] such as lung, prostate, breast, ovarian and head and neck squamous carcinoma, preclinical PNBs improved the diagnostic and therapeutic efficacy and safety [1,2,4,5,7,8,26-32], and minimized a recurrence-caused mortality.

Unmatched “detect & destroy” theranostic capability, instant direct in vivo detection of even single cancer cells and exceptional selectivity and safety of PNBs [1,7,8,26,27] including in humans [33], outperformed other intraoperative in vivo products [1,6-8,26-32] and thus can radically improve cancer surgery outcomes, patients' quality of life and reduce cancer care costs.

However, the clinical applications of PNBs remains limited by the high complexity and costs of the technology. Current in vivo PNBs [1,7,28] require the combination of ultra-short (20-30 ps) near-infrared (NIR) laser pulses and gold nanoparticles (NP). This combination presents several challenges detailed below.

### BRIEF DESCRIPTION OF THE DRAWINGS

FIG. 1 is a diagram of the device and method for the optical generation and detection of PNB with a pump laser 1 with a relatively long pulse duration >200 ps and infrared wavelength, an ultra-low noise continuous probe laser 2 at infrared wavelength different from that of the pump laser, a low-noise high sensitivity photodetector 3 capable of detection the radiation of the probe laser, a fiber optical bundle (a PNB probe) 4 with the several optical fibers for the delivery of the pump laser pulse and probe laser beam to the nanoparticle or the nanoparticle cluster 5, and for the collection of the probe laser light on the same side as the laser beams were delivered from, when the light of the probe laser is scattered by a PNB 6, such scattered light collected by the optical fibers 4 and delivered to the photodetector 3.

FIG. 2 is an image of a fiber optical bundle (1) (a PNB probe) as it generates and detects PNBs in the water suspension of NPs (2).

FIG. 3 is the signals registered with the photodetector connected to the optical fiber bundle in water samples in response to single pump laser pulses of the identical fluence, with identical volume concentration of 50 nm Au and 50 nm TiN NPs.

FIG. 3A is the signals registered in pure water, laser pulse 1064 nm, 325 ps. FIG. 3B is the signals registered in pure water, laser pulse 532 nm, 260 ps.

FIG. 3C is the signals registered in TiN NP suspension, laser pulse 1064 nm, 325 ps.

FIG. 3D is the signals registered in TiN NP suspension, laser pulse 532 nm, 260 ps.

FIG. 3E is the signals registered in Au NP suspension, laser pulse 1064 nm, 325 ps.

FIG. 3F is the signals registered in Au NP suspension, laser pulse 532 nm, 260 ps. A spike in the signal front in A, C, E and F is an artifact unrelated to the signal. X axis—the time after the laser pulse, in us, Y axis—the signal amplitude, in mV.

FIG. 3G is the signals obtained in response to single 1064 nm/325 ps pump laser pulses of 7 uJ: water (dotted line), 50 nm Au suspension (dashed line), 50 nm Tin suspension (solid line), identical concentration of 2 10¹⁰ NP/mL.

FIG. 3H is the signals obtained in response to single 532 nm/260 ps pump laser pulses of 7 uJ: water (dotted line), 50 nm Au suspension (dashed line), 50 nm Tin suspension (solid line), identical concentration of 2 10¹⁰ NP/mL.

FIG. 4A is the dependence of the signal lifetime upon the pump laser pulse energy at 1064 nm, 325 ps, as measured in water suspension of TiN NPs at the concentration 2 10¹⁰ NP/mL. A solid line is a linear fit, its intersection with X axis (energy) shows the PNB generation energy threshold.

FIG. 4B is the dependence of the signal amplitude upon the pump laser pulse energy at 1064 nm, 325 ps, as measured in water suspension of TiN NPs at the concentration 2 10¹⁰ NP/mL. A solid line is a linear fit, its intersection with X axis (energy) shows the PNB generation energy threshold.

FIG. 5 is the PNB lifetime (a metric that quantifies the energy efficacy of PNB generation) measured in water and tissue as function of the NP type (sample) and pump laser wavelength. Control: pure water, Gold 50:50 nm Au NPs in water at 2 10¹⁰ NP/mL, TiN 50:50 nm TiN NPs in water 2 10¹⁰ NP/mL. Hollow squares: 532 nm/260 ps pump pulses, water, solid rhombs: 532 nm pump pulses in tissue, hollow circle: 1064 nm/325 ps pump pulses in water, solid star: 1064 nm/325 ps pump pulses in tissue. Averaged values are shown (n>=8).

FIG. 6 is an image of a fiber optical bundle (a PNB probe) 1 placed on a tissue surface 2 to optically and remotely generate and detect PNBs in the tissue model.

FIG. 7A is the photodetector output signals obtained in NP-treated tissue in response to a single pump laser pulse of identical energy for 1064 nm pulse, intact tissue.

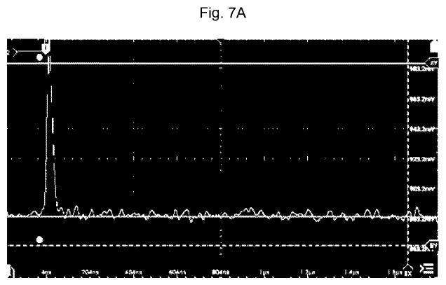

FIG. 7B is the photodetector output signals obtained for 532 nm pulse, intact tissue, false-positive, a false-positive signal is present.

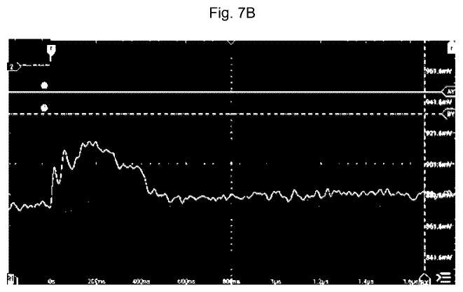

FIG. 7C is the photodetector output signals obtained in NP-treated tissue in response to a single pump laser pulse of identical energy for 1064 nm pulse, TiN NP-treated tissue.

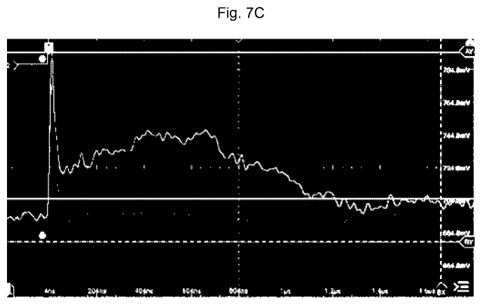

FIG. 7D is the photodetector output signals obtained in NP-treated tissue in response to a single pump laser pulse of identical energy for 532 nm pulse, TiN NP-treated tissue.

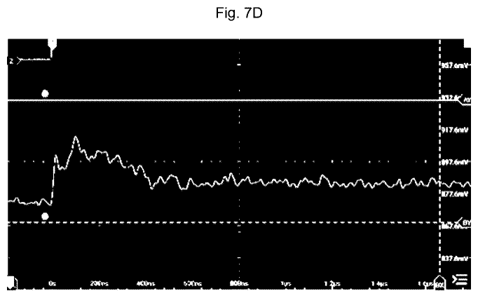

FIG. 7E is the photodetector output signals obtained in NP-treated tissue in response to a single pump laser pulse of identical energy for 1064 nm pulse, Au NP-treated tissue.

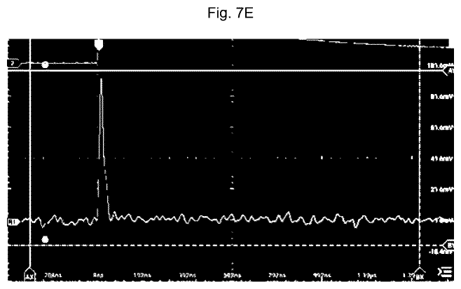

FIG. 7F is the photodetector output signals obtained in NP-treated tissue in response to a single pump laser pulse of identical energy for 532 nm pulse, Au NP-treated tissue.

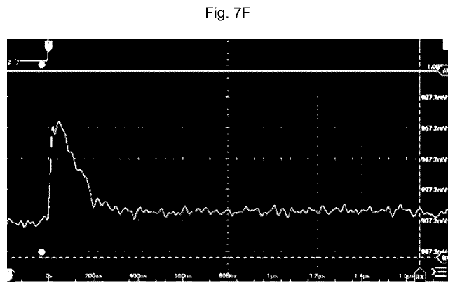

Signals in FIGS. 7A, 7C, and 7E include an artifact spike in the front, unrelated to the signal. The energy of laser pulses was the same as for water suspensions, 7 uJ.

FIG. 8 is the signal amplitude sensitivity parameter S for the PNB signal detection in water and tissue for NP suspension (2 10¹⁰ NP/mL) of 50 nm Au NPs and TiN NPs, and for intact and NP-treated tissue. The probe beam wavelength 1550 nm, power lo 2.5 mW. Hollow squares: 532 nm/260 ps pump pulses, water, solid rhombs: 532 nm pump pulses in tissue, hollow circle: 1064 nm/325 ps pump pulses in water, solid star: 1064 nm/325 ps pump pulses in tissue. Averaged values are shown (n=8).

FIG. 9 is the specific pattern of optical fibers in the fiber bundle (a PNB probe) enables a high-sensitivity optical detection of PNBs: The probe beam delivery fiber of the type 2, single-mode, low numerical aperture, in the center (1), two pump laser pulse delivery fibers of the type 1 (2), multimode, as close as possible to the fiber 1, sixteen fibers, type 4, for the probe laser light collection (3), placed around the delivery fibers.

FIG. 10 is an image of an optical fiber bundle (a PNB probe) 1 as shown inserted into a standard biopsy needle 2.

FIG. 11A is a diagram of the beam delivery into the outer volume outside the PNB probe: (1) a probe laser beam, (2) a pump laser beam(s), (3) a ball lens, (4) a volume outside of the PNB probe where the overlap of the probe and pump laser beams was achieved, so to support the generation and detection of PNBs

FIG. 11B is the optical intensity profiles of the pump laser beams delivered through the delivery optical fibers, one fiber.

FIG. 11C is the optical intensity profile of the pump laser beam delivered through an opposite delivery fiber.

FIG. 11D is the optical intensity profile of the pump laser beam delivered through the two delivery fibers (D), white crosslines showing the center of the probe laser beam.

FIG. 11E is the optical intensity profile of the two pump laser beams, beam 1 and beam 2 and a probe laser beam 3, and shows their good spatial overlap in the volume outside of the fiber optical bundle, with the probe beam 3 illuminating the volume under the excitation with the two overlapping pump laser beams 1 and 2.

### DETAILED DESCRIPTION OF THE INVENTION

The laser-related challenge of the PNB technology is that in vivo-and PNB-optimized lasers use solid-state mode-locked parametric designs that are complex, bulky and costly, cannot be used with standard optical fibers thus limiting surgical in vivo use (Table 1). Such laser pulses can be delivered only with rigid and hence bulky optical guides because high optical intensities of such ultra-short laser pulses exclude the use of standard optical fibers with solid core.

The gold NP-related challenge of the PNB technology is that In vivo- and PNB-optimized gold NPs, for example, nanoshells (Table 2) are thermally damaged (melt) by laser pulses before they convert that pulse into a PNB [34-36] and thus fail to efficiently generate PNBs unless using ultra-short laser pulses [7,36]. Such NPs are often too large for cancer cell targeting (which requires NPs<100 nm) [37-39], with costly and complex manufacturing.

These limitations of the current short laser pulse-gold NP combination drives the costs and complexity of PNB technology and limit its clinical translation. Therefore, the clinical PNB product requires simple and reliable pulsed laser and plasmonic nanoparticles that can be productized and used by physicians and surgeons with standard clinical tools and procedures.

The laser solution. Physics and laser industry translate above requirements into (1) fundamental infrared laser wavelength, like 1064 nm, which the safe for biological tissue and penetrates deep into biological tissue, (2) laser pulse duration >200 ps, to reduce the optical intensity and enable optical delivery via flexible standard optical fiber with a solid core (3) the reduced size, complexity and cost of the laser. A solution is a compact and low-cost microchip laser that delivers 200-500 ps pulses at infrared wavelength, for example, at 1064 nm (Table 1), through a silica optical fiber. Such laser pulses, however, need matching plasmonic NPs to efficiently convert longer laser pulses into PNBs without increasing laser energy doses above biologically safe levels.

TABLE 1
Lasers for PNB generation

| | Current | Solution |
|---|---|---|
| Pulse duration, ps | 20-30, cannot be delivered via a | 200-500, can be delivered via a |
| | standard solid optical fiber | standard solid optical fiber |
| Laser wavelength | 700-800, need to convert a | 1064, fundamental wavelength, |
| (nm) and design | fundamental wavelength, a | microchip compact laser |
| | parametric mode-locked laser | |
| PNB in vivo device | Free-space rigid bulky PNB | Flexible optical fiber probe of |
| | probe | microscopic size |
| Laser device weight, | 50-100 | <1 |
| kg | | |
| Cost, $ | 130,000-200,000 | 8,000-15,000 |

The nanoparticle solution (Table 2). Recently developed ceramic nanomaterials, transition metal nitrides, shift plasmonic optical absorption from visible range (typical for gold NPs) towards 1000 nm for smaller NPs with the size below 100 nm [40-48].

Among such materials, titanium nitride (TiN) NPs, demonstrated a combination of clinically- and PNB-important properties (Table 2): (1) biocompatibility [39,43,44,46-48], (2) small size, 20-80 nm, to support a cancer cell NP targeting, internalization and clustering

TABLE 2
Nanoparticles for in vivo PNB generation

| | Current | Solution |
|---|---|---|
| Material | Gold | Titanium nitride |
| Size, nm | 200-240 | 40-80 |
| Cell level targeting | Limited (too | Good |
| and clustering in vivo | large NPs) | |
| Biocompatibility | Good | Good |
| Melting temperature (loss | ≈1000 | ≈3000 |
| of PNB generation), C. | | |
| PNB energy efficacy at longer | Low, NP melts | High, NP does |
| laser pulse >200 ps | | not melt |
| Cost, dry weight, per gram, $ | 200-400, | 2-4, multiple sources |
| | limited sources | |

in vivo [39-41,48]; (3) good plasmonic performance at 1064 nm for NPs of 20-80 nm size [40-48], (4) high thermal stability and laser damage threshold of TiN NPs (TiN melting temperature is 3-fold higher than that for gold) allows such NPs to efficiently convert a longer laser pulse into a PNB without losing its plasmonic properties due to laser-induced thermal damage to NP [42,44,49-50], and (5) low cost and well-established manufacturing.

Thus, the plasmonic and thermal properties of TiN NPs can radically improve PNB generation in vivo with longer infrared laser pulses of microchip lasers delivered through standard optical fibers. However, a microchip laser-TiN NP combination neither all-fiber optical PNB generation and detection were not realized yet for PNB generation and in clinical applications.

A long laser pulse—TiN NP—fiber optical PNB generation and detection concept was verified with the prototype and experimental data described below.

The pump lasers of microchip type, with relatively long pulses with duration of hundreds of picoseconds, 260 ps at 532 nm and 325 ps at 1064, were employed.

The pump laser pulses were delivered to the sample via a standard solid core optical fiber.

The laser pulses were coupled into optical fibers on proximal end of the fiber optical bundle, and were collimated with additional optics on a distal end of such bundle, in contact with the sample, so to maintain an approximately permanent level of the pump laser fluence in the sample near their entry into the sample.

The NPs that converted laser energy into PNBs were titanium nitride (TiN) NPs of 50 nm size (Table 3). As the reference plasmonic NPs, gold (Au) NPs of the same size, 50 nm, were used since we well established PNB generation with Au NPs [6].

TABLE 3
Properties of the nanoparticles for
PNB generation with long laser pulses

| NP material | TiN | Au |
|---|---|---|
| Size, nm | 50 | 50 |
| Structure and shape | Solid cubes and spheres | Solid spheres |
| Concentration in | 2 10¹⁰ | 2 10¹⁰ |
| suspension, NP/mL | | |
| Optical density in suspension, | 0.03 (1020) | 0.6 (530) |
| a.u.(wavelength, nm) | | |

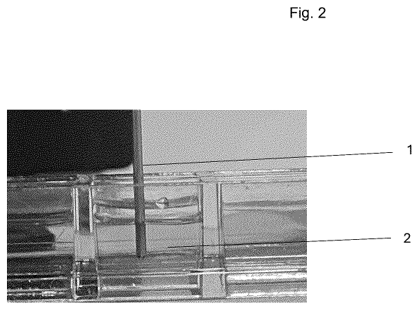

PNBs were detected on the same side of the sample as the pump and probe laser beams entered the sample, by using the optical back-scattering of a probe laser beam at 1550 nm, delivered to and collected from the sample with an optical fiber bundle (FIG. 2). A continuous ultra-low noise laser has been coupled into the optical fiber.

Multimode optical fibers were used to collect the back-scattered by a PNB light of a probe laser and deliver the collected light to an amplified photodetector.

Individual electrical signals, in response to single pump laser pulses, were registered with a digital oscilloscope synced with a pump laser pulse.

The two PNB metrics have been derived from the signal:
the efficacy of PNB generation was quantified with a PNB lifetime, the duration of a PNB-positive signal. We have established previously [1-6] that such metrics characterizes the maximal diameter of the PNB which, in turn, describes the PNB generation energy efficacy
the sensitivity of the optical detection of PNBs was quantified with the arbitrary signal amplitude S, and compensated for a signal noise, the power of the probe laser beam and the photodetector conversion gain:

$$S = {( {V}^{2} - {V}_{noise}^{2} )}^{0.5} / Gain {I}_{0} ,$$

where V (mV) is the amplitude of the signal, Vnoise (mV) is the amplitude of the noise (at the absence of the pump pulse), Gain is the conversion of the photodetector (mV/uW) and I₀ is the power of the probe laser beam (uW). This metric described the sensitivity of the PNB detection.

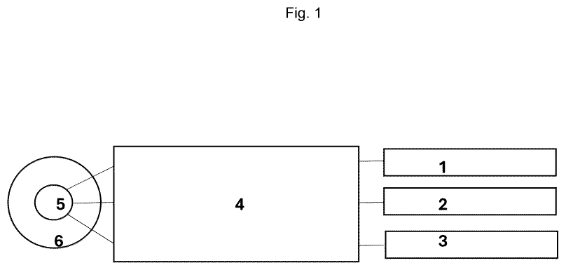

A prototype device included several components: a pump pulsed laser, and continuous probe laser, a photodetector, optical fiber bundle for the delivery of optical energy to/from the sample, an oscilloscope and the laser beam characterization hardware (FIG. 1).

The prototype device (FIG. 1) was applied to optically generate and detect PNBs in water suspension of TiN and Au NPs (of identical volume concentration of 2 10¹⁰ NP/mL) and in response to single laser pulses at specific wavelength and fluence. The fiber optical bundle was submerged into the water suspension of NPs to generate and detect PNBs at the tip of the device (FIG. 2).

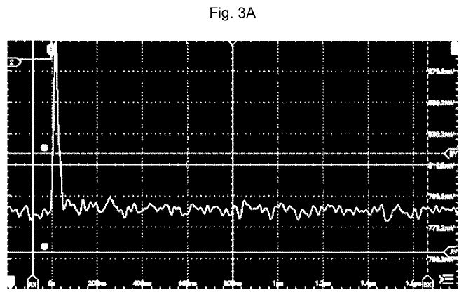

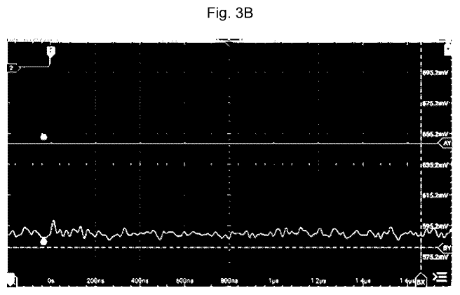

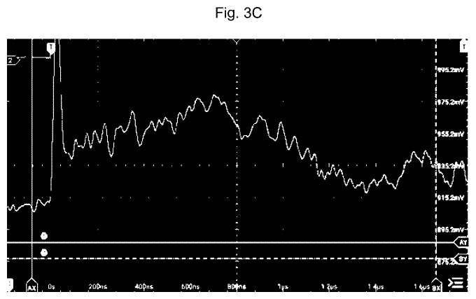

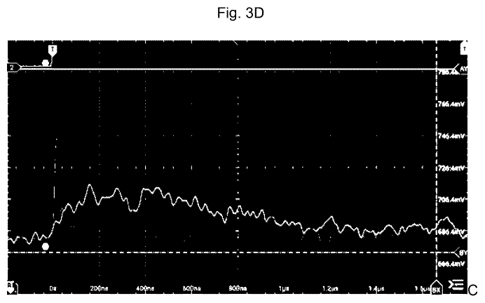

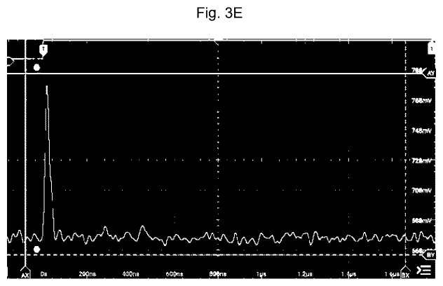

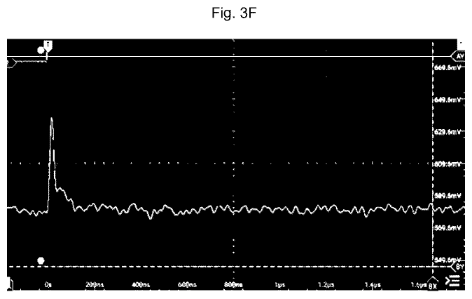

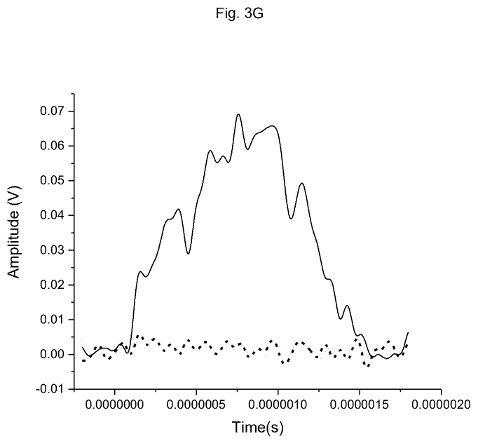

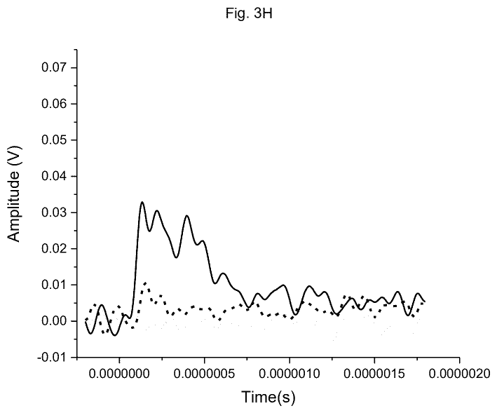

A PNB-positive optical scattering signal (FIG. 3, C, D) was formed by the expansion (the signal front) and collapse (the signal tail) of a vapor-water boundary of the PNB. In case of multiple simultaneously generated PNBs of various maximal size, a typical case in a suspension of NPs with an average inter-particle spacing around 4 um, durations of PNBs may differ significantly, resulting and a sharp front and delayed tail.

At 1064 nm, an artifact signal, a spike, was present due to the poor filtering of the pump laser pulse at the photodetector. That spike was ignored while measuring the signal metrics.

PNB-positive signals were observed around Au (FIG. 3 F) and TiN (FIG. 3 D) NPs under 532 nm visible excitation.

PNB-positive signals were observed only around TiN NPs under 1064 nm infrared excitation with longer pump laser pulses (FIG. 3 C, E).

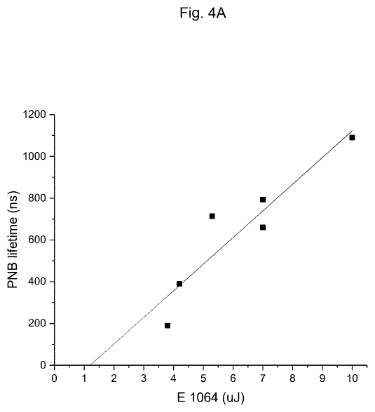

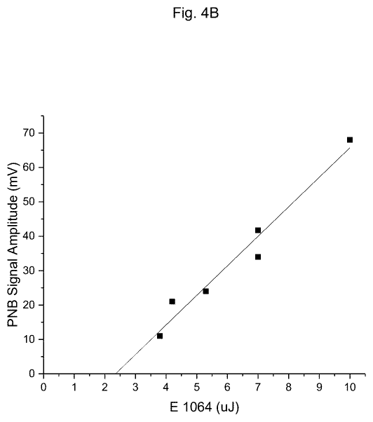

To ensure correct conditions for experimental analysis of the PNB generation, the PNB generation energy threshold for TiN NPs was determined. This was achieved through measuring the PNB lifetime and signal as function of the pump laser fluence (FIG. 4). The pump laser wavelength of 1064 nm was used since it corresponded to were their plasmonic properties promise a good performance [40-48] and at the same time, their intended biomedical use with the minimal biodamage and the maximal tissue optical penetration depth [39,43,44,46-48], all compared to 532 nm.

To estimate the PNB generation threshold at 1064 nm, the linear data fits (black line, FIG. 4) were extrapolated to zero signal (no PNBs). Both types of metrics yielded similar thresholds around 30-40 mJ/cm² at 1064 nm (the linear fits resulted in 23 mJ/cm² (a lifetime plot) and 42 mJ/cm² (the signal plot).

At 532 nm, close to their plasmon resonance, we previously determined the PNB generation threshold for Au NPs, solid spheres of similar size and under similar conditions (water suspension, a pump pulse duration of hundreds ps), to be 115 mJ/cm2 [36].

At that infrared wavelength of 1064 nm, no PNB generation was detected for Au NPs. Such inability of Au NPs (solid spheres used in the study) to generate PNBs in the infrared corresponded well to their plasmonic and PNB properties for that wavelength and duration of the pump pulse as we measured previously [6].

The signal metrics of PNB generation in water suspensions were experimentally compared for Au and TiN NPs at the two wavelengths, 532 nm and 1064 nm. The pump wavelength of 532 nm was close to plasmon resonance of Au NPs, where their PNB and plasmonic properties were well-established [6,36].

The infrared pump wavelength of 1064 nm matched three requirements:
1. Biocompatibility: at that wavelength, the optical absorbance in tissue and blood and hence a laser pulse-induced biodamage are the lowest;
2. That wavelength matches spectral region of the maximal optical absorbance of TiN NPs;
3. That wavelength matches the laser industry standards as the fundamental harmonic of many designs for pulsed lasers.

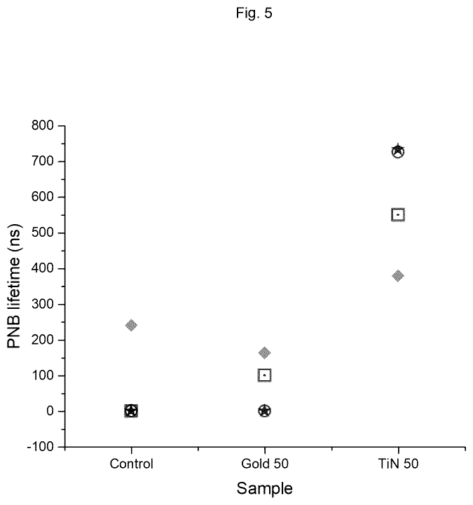

PNB lifetimes, as metrics of PNB energy efficacy, were obtained for single laser pulses under identical fluence of 126 mJ/cm², NP size of 50 nm and volume concentration of 2 10¹⁰ NP/mL, and in three samples: pure water, Au NP suspension, TiN NP suspension (FIG. 5).

Both wavelengths of laser pulses produced no PNBs detected in pure water. Comparison of the signals obtained at 532 nm and 1064 nm for Au vs TiN water suspensions revealed several important properties of the PNB generation (FIG. 5):
1. Au NPs generated PNB at the wavelength close to their plasmon resonance, 532 nm, and did not generate PNBs under infrared excitation at 1064 nm. This result was in line with our prior experimental results for Au NPs pumped with laser pulses of several hundred picosecond duration at 532 nm [6,36];
2. TiN NPs generated PNBs under both visible and infrared excitation, with the highest PNB generation efficacy achieved under the infrared pumping at 1064 nm;
3. The energy efficacy of PNB generation by TiN NPs at 1064 nm was more than 7-fold higher than that for Au NPs at 532 nm under similar duration of the pump laser pulse. This result proved the superior PNB properties of TiN NPs compared to those of Au NPs.

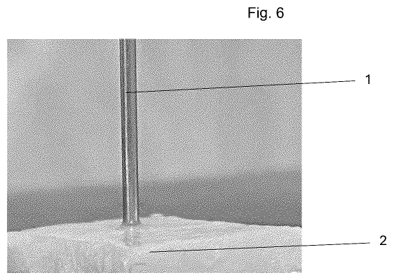

The process of PNB generation was also studied in a tissue model, with the suspensions of the same concentration of NPs, as studied above, stained into the surface of the tissue slices. An optical fiber bundle was brought in contact with the wetted tissue surface and single pump laser pulses were applied in several different locations (FIG. 6).

The signals were obtained with a fiber bundle at contact with the tissue surface and in response to single pump laser pulses (FIG. 5).

The PNB generation probability (Table 4) and average lifetimes (FIG. 5) were measured at 126 mJ/cm2, same pump laser fluence as for the water suspensions (with the exception for TiN NPs where the fluence was reduced to 94 mJ/cm2, in order to prevent the mechanical damage of the fiber lens surface by too large PNBs).

TABLE 4
The probability of PNB generation in a tissue model

| Sample, pump | | | |
|---|---|---|---|
| laser wavelength | NP-free control | 50 nm Au NPs | 50 nm TiN NPs |
| Water, 532 nm | 0 | 64 | 83 |
| Water, 1064 nm | 0 | 0 | 100 |
| Tissue, 532 nm | 63 | 88 | 100 |
| Tissue, 1064 nm | 0 | 0 | 43 |

In tissue, a visible pump laser wavelength (532 nm) was absorbed even by intact tissue (without any NPs) and such intact tissue occasionally generated vapor bubbles, due to residual optical absorption of the laser pulse by hemeproteins. This result was in line with well-established fact that 532 nm laser pulses are not safe for live tissues. NP-treated tissue generated PNBs but also with less than 100% probability because of non-uniform distribution of NPs in the near-surface volume of the tissue. As in the water suspensions, TiN NPs were more efficient than Au NP for the PNB generation.

Switching to the infrared wavelength (1064 nm) eliminated “false-positive” PNB-like signals in intact tissue. At the same time, TiN NP-treated tissue generated the largest PNBs with the highest PNB lifetime. Even under the reduced pump laser fluence (74% of that used for 532 nm pulses and for water model) the PNB lifetime was close to that of PNBs generated in TiN NP water suspension under the higher pump laser fluence. At the same time, no PNBs were generated in Au NP-treated tissue.

For TiN NPs, the infrared pump laser pulse wavelength matched both requirements of biosafety and high energy efficacy of PNB generation.

Based upon PNB generation threshold and lifetime data obtained and on our previous data for Au NP-generated PNBs [6,29,36], TiN NPs provided a multi-fold improvement in the energy efficacy of the PNB generation with the infrared and relatively long laser pulses, as compared to Au NPs of the same size.

Further, such high PNB generation efficacy was achieved at the infrared wavelength of 1064 nm which is the most advantageous in terms of both the biomedical applications of PNBs and the pulsed laser design for PNBs.

From the laser industry standpoint, 1064 nm is the fundamental harmonic available in many laser designs. This, in turn, allows to use low-cost, compact and simpler pump lasers compared to those required for PNB generation with Au NPs.

The longer duration of the pump laser pulse, hundreds of picoseconds, instead of 20-30 ps as the optimal pulse duration for Au NPs, resulted in much lower optical intensity. The reduced optical intensity, in turn, enabled the use of standard (solid) optical fibers for a minimally invasive delivery of pump laser pulses, unlike bulky rigid and complex delivery guides required for high-intensity picosecond laser pulses required for Au NP-generated PNBs. This optical fiber delivery option opens a new range of opportunities for in vivo generation of PNBs with minimally invasive microscopic fiber optical probes, a solution technically impossible for 20 ps laser pulses required for Au NP-generated PNBs [6,36].

Splitting the pump pulse over the two delivery optical fibers has slightly reduced the PNB generation efficacy, as the PNB lifetime decreased from 755 ns (one delivery fiber) to 467 ns (same energy delivered via the two fibers). A single delivery fiber for a pump laser pulse is therefore an optimal design solution.

Averaging the PNB signal over several pump laser pulses did not improve the signal-to-noise ratio (despite reducing the noise): each next consecutive pump laser pulse returned a smaller PNB so that the multi-pulse average PNB lifetime was approximately half of the lifetime of the PNB generated by the 1^{st} laser pulse. Such decay may be attributed to the water suspension model where NPs are mechanically pushed out of the actively pumped volume by each previous PNB.

The optical detection of PNBs was performed on the same side of the sample as the pump and probe laser beam were delivered from, and has used the same optical fiber bundle as for the delivery of the pump and probe laser beams (FIG. 1).

To detect PNBs optically, the back-scattering of a low-noise probe laser beam has been applied. Elastic optical backscattering by PNBs, especially in the high background of biological tissue, requires the combination of a stable, safe and low-noise probe laser beam with a high-sensitivity and low noise phot-detector. Such combination was realized at 1550 nm which is biologically safe, with a probe laser delivering light through an optical fiber in milliwatt range, and the back-scattered light collected by other standard optical fibers coupled to a photodetector with the gain in the range 20-1600 mV/uW.

The sensitivity of the optical detection of PNBs with a standard optical fiber was quantified and experimentally analyzed in water and tissue through the signal metric S that compensated for the power of the probe laser, the signal noise and the gain of the photodetector.

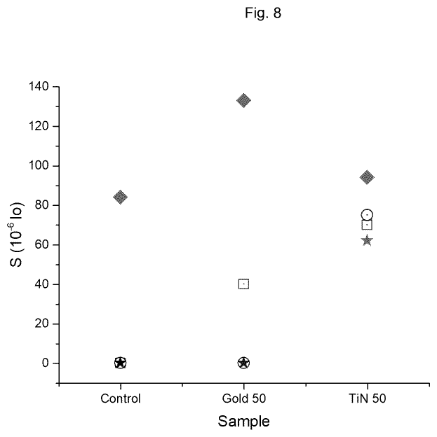

Such a metric S directly characterized the sensitivity of the optical detection of the PNBs generated in two types of samples (water suspensions and tissue) and by the two types of nanoparticles of the same size, Au and TiN NPs (FIG. 8).

Au NP-generated PNBs yielded relatively high signal amplitudes, both in water and in tissue, under 532 nm pump laser pulses (FIG. 8).

However, at that pump laser wavelength of 532 nm, an intact tissue (shown as “control” in FIG. 8) also returned signals of significant amplitude. Such “false-positive” signals disqualify visible wavelengths for PNB generation in vivo, regardless the sensitivity of PNB detection. Under infrared excitation at 1064 nm, no PNB signals were detected in water and tissue samples with Au NPs.

TiN NP-generated PNBs yielded the highest optical signal amplitudes under infrared excitation at 1064 nm (FIG. 8). In water, such signals showed an absolute maximum in the detection sensitivity.

In tissue samples under excitation with visible wavelength the detection sensitivity of TiN NP-generated PNBs was comparable to that for Au-generated PNBs. However, the PNB specificity of such Au NP-generated PNB signals at 532 nm was low, since similar signals were detected in the NP-free intact tissue.

This was an infrared excitation wavelength of 1064 nm that brought the high PNB signal specificity of the detection, with the high contrast between TiN Np and intact samples, both for water (80:0) and tissue (60:0).

These experiments proved a new concept of the optical generation and detection of PNBs with a novel combination of (A) low-cost simple microchip pulsed laser in infrared, with biologically safe and relatively long laser pulses that are safe for biological tissues, (B) thermally-resistant and low-cost commercially available TiN ceramic nanoparticles of small size which ensures their biocompatibility and efficient in vivo targeting, and internalization and clustering by target cell and (C) all-fiber optical generation and detection of PNBs with standard optical fibers in a minimally invasive way.

Such long infrared laser pulse—small TiN NP—fiber optical bundle combination solved several limitations of PNB technologies that rely on gold and other noble metal NPs.

Specifically, a laser pulse-induced thermal damage of noble metal NPs requires very short laser pulses, in the range 20-30 ps [6,7, 36].

These innovations for the PNB generation are summarized in the tables 1 and 2, for the lasers and for the nanoparticles, respectively.

In biomedical and clinical applications of PNB technology, longer laser pulses allow to switch from free-space optical guides which are bulky and often invasive rigid (such as rigid endoscopes or optical arms) to flexible standard optical fibers this reducing the size of the optical guide by 10-100 times and improving the flexibility and accessibility in vivo, not to mention a compatibility with standard clinical tools.

Further, all-optical fiber PNB technology, without relying upon electrical signals which are susceptible to electric and magnetic field associated with clinical tools (MRI, CT), can be used with such clinical equipment without compromising the PNB generation and detection.

The fiber optical bundle (“PNB probe”) if used in the clinic can be a single-time use device.

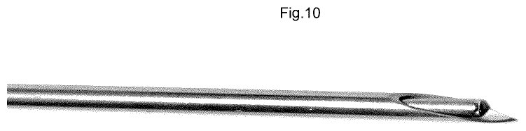

All-optical generation and high-sensitivity detection of PNBs in a minimally invasive way requires to combine several requirements into one design: (1) small diameter and high flexibility of the fiber bundle, less than 2 mm, to route it through standard biopsy needles, endoscopes and other clinical tools (FIG. 10), (2) the maximal optical collection of the probe laser light scattered and back-scattered by PNBs, (3) the minimal optical background caused by the optical scattering or back-scattering of the probe laser light by the sample media, for example, biological tissue and (4) biocompatibility of the part of the fiber bundle which is in contact with a patient or biological tissue.

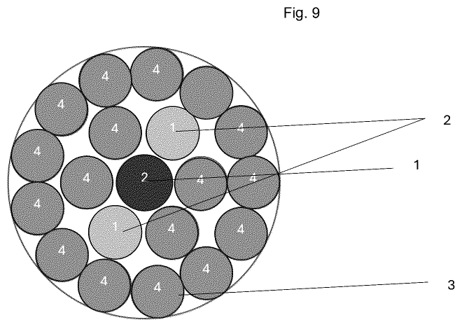

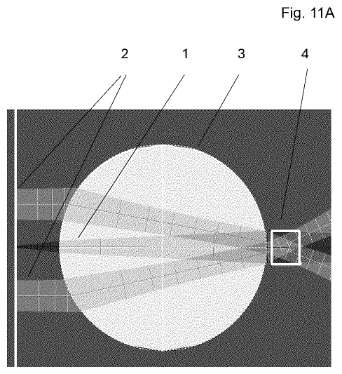

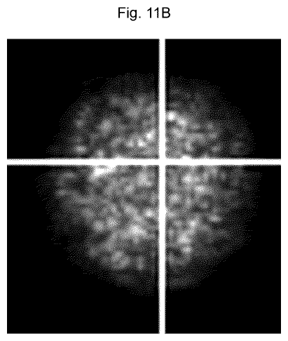

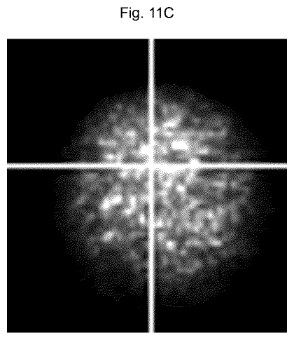

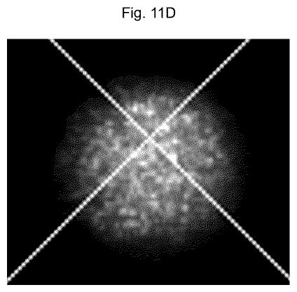

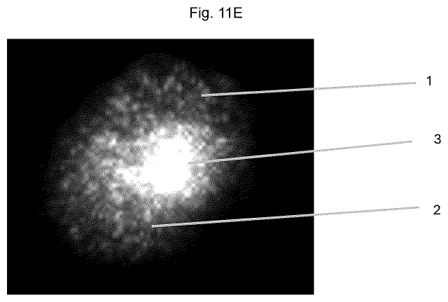

All four requirements were realized in the design of the optical fiber bundle (also referred to as a “PNB probe”) which included the combination of (FIG. 9):
a central single-mode optical fiber (#1, FIG. 9) to deliver the probe laser beam so to minimize the optical background, such fiber connected to the probe laser on its proximal end;
multi-mode low numerical aperture optical fibers (#2, FIG. 9), from one to four, placed around the probe laser delivery fiber, to deliver a pump laser pulse into the tissue volume illuminated by the probe laser, such fibers optically coupled to a pump laser on their proximal end;
several peripheral optical fibers (#3, FIG. 9) of high numerical aperture, >0.2, to collect PNB-scattered and-backscattered probe laser light and deliver such light to the photodetector, such fibers optically coupled to the photodetector on their proximal end;
the distal tips of all above fibers placed at specific distance, for example 20 um to 150 um, from a sapphire ball lens of the diameter from 0.5 mm to 0.9 mm, such lens being in contact with the tissue or sample and supporting the delivery and collection of the optical energy to/from the PNB-generating NPs in the sample (FIG. 11a);
all said fibers and lens enclosed into a biocompatible casing, for example, a metal tube of the outer diameter compatible to biopsy needles or other clinical tools employed for diagnostic, therapeutic or surgical procedures;
all said fibers are standard silica fibers.

A ball lens was in contact with the sample, so to maintain an approximately permanent level of the pump laser fluence in the sample near the beam entry into the sample. The delivery of the pump and one probe laser beams was directly tested with the glassless image sensor (FIG. 11 b,c,d,e). Images of the pump beam (FIG. 11 b,c,d,e) were used to measure the beam diameters, at specific distances from the ball lens, so to calculate the fluence of the pump laser pulse and the intensity of the probe laser beam. The pump laser beam remained nearly collimated within the distance 0-100 um from the ball lens. A good overlap between a probe and pump beams was found in the distance range from the ball lens from 0 um to 70 um.

The said fiber bundle was prototyped as a PNB probe compatible with a biopsy needle (FIG. 10). the example of the design specifications of the said optical fiber bundle and PNB probe for all-optical minimally-invasive generation and detection of PNBs is shown in the table 5.

TABLE 5
Design specifications for a fiber optical bundle (a PNB probe)

| | | | | |
|---|---|---|---|---|
| Specification | Unit | Target | Min | Max |

Distal, the part that goes into the needle

| Outer diameter | mm | | | 1,050 |
|---|---|---|---|---|
| Length | mm | | 51 | 55 |
| Ball lens, sapphire, | mm | 0.7 | | |
| diameter | | | | |
| Ball lens installation | The inner back surface clean and free of debris | | | |
| Lens extend ended | mm | | 0.25 | 0.35 |
| height, out of the tubing | | | | |
| Lens mounting in the | | Waterproof, | | |
| tube | | shockproof | | |
| Gap between the lens | um | 50 | 40 | 70 |
| surface and the fiber tip | | | | |

| | |
|---|---|
| Materials | Sapphire lens, ss tube, epoxy |
| Optical fiber pattern | The diagram: SMF precisely in the center, two delivery MMF |
| | as close as possible to SMF, symmetrically opposite, collection |
| | MMFs in two rows |
| Central fiber #1, probe | SMF, 1550 nm wavelength, NA 0.12 9/125/145 THORLABS |
| beam delivery, 1 pc | SINGLE MODE FIBER SM1550P, position in the center of |
| | the tip and ball lens, without buffer |
| Side fiber #2, pump | MMF, step index, 1064 nm, 105/125 THORLABS NA = 0.10, |
| beam delivery, 2 pc | FG105LCA, without buffer, position symmetrically and |
| | opposite each other, relative to the SMF central fiber |
| Peripheral collection | MMF, 1550 nm wavelength, step index, 105/125/250 |
| fibers #3, 16 pc | THORLABS NA = 0.22, without buffer |
| Distal tip | Polished, cleaned and free of debris |

Back end

| Materials | Ss tube, soft epoxy, jacket for the full length of the fibers | | | |
|---|---|---|---|---|
| Length | mm | | 35 | 45 |
| Outer diameter | mm | 1.6 | | |
| Transition from the | Waterproof, soft elastic epoxy | | | |
| distal tube to the back | | | | |
| tube | | | | |
| Extend to the fiber | | | | |
| jacket | | | | |

Fiber bundle

| Length of the fiber, total | mm | 2000 | | |
|---|---|---|---|---|
| Distal fiber tip | Patterned | | | |
| | and | | | |
| | polished | | | |
| Length of the fiber | mm | 1700 | | |
| bundle | | | | |
| Length of individual | mm | 300 | | |
| proximal legs | | | | |
| Difference in the total | mm | | | 3 |
| length of the fibers 2, | | | | |
| distal tip to ferule tip | | | | |
| Jacketing | From the back tube to proximal connectors, black | | | |
| Jacket diameter, the | mm | 2.3 | | |
| bundle | | | | |
| Jacket diameter, | Individual jackets HYTREL TUBING 900 UM DIA - BLACK | | | |
| Proximal legs | | | | |
| Connectors, proximal | | | | |
| legs | | | | |
| Leg 2 | FC/PC, | | | |
| | ceramic | | | |
| Leg 1 | FC/APC, | | | |
| | ceramic | | | |
| Leg 3 | FC/PC, | | | |
| | metal | | | |
| Leg 2 | A bundle of 16 fibers w/o buffer. The minimal optical diameter | | | |
| Marking | Each connector with the leg # | | | |
| Optical transmission, | % | | 89 | |
| Leg 1 | | | | |
| Optical transmission, | % | | 89 | |
| Leg 2 | | | | |
| Optical transmission, | visual | | | |
| Leg 3 | | | | |

The said optical fiber bundle (PNB probe) was built to co-deliver the pump and probe laser beams, with their efficient overlap in the space outside the said bundle (FIG. 11).

To summarize, a combination of a microchip laser with longer infrared pulses, small TiN NPs and flexible optical fiber bundle supported the novel all-fiber optical PNB technology, method and device for a minimally-invasive generation and detection of PNBs is samples which are outside of the PNB device and can be used for detecting and treating diseases in vivo with PNBs for the minimally invasive medical diagnostics, therapy and surgery.

The improvements include but are not limited by replacing expensive and bulky picosecond mode-locked lasers and thermally fragile nanoparticles gold NPs with compact low-cost lasers and low-cost biocompatible titanium nitride NPs.
One of the most impactful clinical applications of that novel a long laser pulse—TiN NP—optical fiber concept relates to cancer biopsy and surgery. With their direct, instant and minimally invasive in vivo detection (and optional destruction) of microscopic primary or residual tumors at the cell-level (otherwise undetectable or unresectable), PNBs, integrated with standard diagnostic or surgical tools and procedures, will create the following impacts:
Early precise and minimally invasive cancer diagnosis: (1) real-time high-sensitivity in vivo preliminary diagnosis before extracting the tissue; (2) PNB-guided sampling will make a standard biopsy less invasive and more conclusive; (3) detection of even microscopic tumors will enable early-stage treatment, (4) easy integration of PNB device into standard biopsy procedure; (5) applicability to a broad spectrum of cancers and (6) the automated PNB diagnosis reduces the dependence upon human expertise. Broader impacts include shorter time to treatment, thus improving clinical outcomes and reducing cancer care complexity and cost;
Recurrence-free and minimally invasive PNB-assisted cancer surgery [7]: (1) will help surgeons to guide the resection of microscopic residual tumors thus improving surgical outcomes and quality of life, (2) reduce the duration and cost of cancer surgery, (3) reduce the cost and toxicity of post-operative treatment;
The universal mechanism and unique “detect & destroy” theranostic capability of PNBs, unmatched by other in vivo real-time technologies, enables their future use in multiple cancers, as we demonstrated previously [1-8,26-36].

The universal mechanism and unique “detect&destroy” theranostic capability of PNBs, unmatched by other in vivo real-time technologies, enables their future use in multiple cancers, as we demonstrated previously [1-8,26-36].

The references to the foregoing paragraphs regarding previous disclosures are as follows:
1. Lukianova-Hleb EY, Ren X, Sawant RR, Wu X, Torchilin VP, Lapotko DO. On-demand intracellular amplification of chemoradiation with cancer-specific plasmonic nanobubbles. Nat Medicine 20:778-784 (2014).
2. Lapotko D, Lukianova-Hleb E, Oraevsky A. Clusterization of nanoparticles during their interaction with living cells. Nanomedicine 2:241-253 (2007).
3. Lukianova-Hleb E, Hu Y, Latterini L, Tarpani L, Lee S, Drezek R, Hafner J, Lapotko D. Plasmonic nanobubbles as transient vapor nanobubbles generated around plasmonic nanoparticles. ACS Nano 4:2109-2123 (2010)
4. Lapotko D, Lukianova E, Oraevsky A. Selective laser nano-thermolysis of human leukemia cells with microbubbles generated around clusters of gold nanoparticles. Lasers Surg Med 38:631-642 (2006).
5. Lapotko D, Lukianova E, Potapnev M, Aleinikova O, Oraevsky A. Method of laser activated nanothermolysis for elimination of tumor cells. Cancer Lett 239:36-45 (2006).
6. Lapotko D. Plasmonic nanobubbles: from physics to clinics. 1^{st} Ed. Jenny Stanford Publishing, pp. 1180: ISBN 9789815129076 (2025).
7. Lukianova-Hleb EY, Kim YS, Belatsarkouski I, Gillenwater AM, O'Neill BE, Lapotko DO. Intraoperative diagnostics and elimination of residual microtumours with plasmonic nanobubbles. Nat Nanotechnology 11:525-532 (2016).
8. Lukianova-Hleb EY, Lapotko DO. Rapid detection and destruction of squamous cell carcinoma of the head and neck by nano-quadrapeutics. Head Neck 37:1547-1555 (2015).
9. Eldeeb H, Macmillan C, Elwell C. Hammod A. The effect of the surgical margins on the outcome of patients with head and neck squamous cell carcinoma: single institution experience. Cancer Biol Med 9:29-33 (2012).
10. Robbins KT, Triantafyllou A, Suárez C, López F, et al. Surgical margins in head and neck cancer: Intra- and postoperative considerations. Auris Nasus Larynx 46:10-17 (2019).
11. Higginson JA, Breik O, Thompson AH, et al Diagnostic accuracy of intraoperative margin assessment techniques in surgery for head and neck squamous cell carcinoma: A meta-analysis. Oral Oncology 142:106419 (2023).
12. Pierik AS, Leemans CR, Brakenhoff RH. Resection margins in head and neck cancer surgery: An update of residual disease and field cancerization. Cancers (Basel) 13(11):2635 (2021).
13. Looser KG, Shah JP, Strong EW. The significance of “positive” margins in surgically resected epidermoid carcinomas. Head Neck Surg 1:107-111 (1978).
14. Vikram B, Strong EW, Shah JP, Spiro R. Failure at the primary site following multimodality treatment in advanced head and neck cancer. Head Neck Surg 6:720-723 (1984).
15. Yuen PW, Lam KY, Chan AC, Wei WI, Lam LK. Clinico-pathological analysis of local spread of carcinoma of the tongue. Am J Surg 175:242-244 (1998).
16. Meier JD, Oliver DA, Varvares MA. Surgical margin determination in head and neck oncology: current clinical practice. The results of an International American Head and Neck Society Member Survey. Head Neck 27:952-958 (2005).
17. Rohde M, Rosenberg T, Pareek M, Nankivell P, Sharma N, Mehanna H, Godballe C. Definition of locally recurrent head and neck squamous cell carcinoma: a systematic review and proposal for the Odense-Birmingham definition. Eur Arch Otorhinolaryngol. 277:1593-1599 (2020).
18. Calabrese L, Ostuni A, Ansarin M, Giugliano G, Maffini F, Alterio D, Rocca MC, Petralia G, Bruschini R, Chiesa F, Association of Radiotherapy and Oncology of the Mediterranean Area (AROME). Future challenges in head and neck cancer: from the bench to the bedside? Crit Rev Oncol Hematol 84: e90-96 (2012).
19. van Houten VM, Leemans CR, Kummer JA, Dijkstra J, Kuik DJ, van den Brekel MW, Snow GB, Brakenhoff RH. Molecular diagnosis of surgical margins and local recurrence in head and neck cancer patients: a prospective study. Clin Cancer Res10(11): 3614-3620 (2004)
20. Wind J, Smit EJ, Senan S, Eerenberg J-P. Residual disease at the bronchial stump after curative resection for lung cancer. Eur J Cardiothorac Surg 32:29-34 (2007).
21. Stojadinovic A, Leung DH, Hoos A, Jaques DP, Lewis JJ, Brennan MF, Analysis of the prognostic significance of microscopic margins in 2,084 localized primary adult soft tissue sarcomas. Ann Surg 235:424-434 (2002).
22. Braakhuis BJ, Bloemena E, Leemans CR, Brakenhoff RH. Molecular analysis of surgical margins in head and neck cancer: More than a marginal issue. Oral Oncol 46:485-491 (2010).
23. NIH/NCI program announcement 452 “Translation of Novel Cancer-Specific Imaging Agents and Techniques to Mediate Successful Image-Guided Cancer Interventions” August 2022 https://sbir.cancer.gov/small-business-funding/contracts/current-solicitation/452
24. Heidkamp J, Scholte M, Rosman C, Manohar S, Fütterer JJ, Rovers MM. Novel imaging techniques for intraoperative margin assessment in surgical oncology: A systematic review. Int J Cancer 149:635-645 (2021).
25. ARPA-H: Broad Agency Announcement “Precision Surgical Interventions” 75N99223R0004 August 2023 https://sam.gov/opp/c17fabdf578b481096aaac0debcbf27b/view
26. Lukianova-Hleb EY, Ren X, Townley D, Wu X, Kupferman M, Lapotko D. Plasmonic nanobubbles rapidly detect and destroy drug-resistant tumors. Theranostics 2:976-987 (2012).
27. Lukianova-Hleb EY, Ren X, Zasadzinski JA, Wu X, Lapotko D. Plasmonic nanobubbles enhance efficacy and selectivity of chemotherapy against drug-resistant cancer cells. Adv Mater 24:3831-3837 (2012).
28. Lukianova-Hleb E, Kim Y-S, Aryasomayajula B, Boulikas T, Phan J, Hung M-C, Torchilin V, O'Neill BE, Lapotko DO. Safety and efficacy of quadrapeutics versus hemoradiation in head and neck carcinoma xenograft model. Am J Cancer Res 5:3534-3547 (2016).
29. E. Lukianova-Hleb, E. Yvon, E. Shpall and D. Lapotko All-in-one processing of heterogeneous human cell grafts for gene and cell therapy. Mol Ther Methods Clin Dev 3:16012 (2016).
30. Hleb EY, Hafner JH, Myers JN, Hanna EY, Rostro BC, Zhdanok SA, Lapotko DO. LANTCET: elimination of solid tumor cells with photothermal bubbles generated around clusters of gold nanoparticles. Nanomedicine 3:647-667 (2008).
31. Lukianova-Hleb EY, Hanna EY, Hafner JH, Lapotko DO. Tunable plasmonic nanobubbles for cell theranostics. Nanotechnology 21:085102 (2010).
32. Wagner DS, Delk NA, Lukianova-Hleb EY, Hafner JH, Farach-Carson MC, Lapotko DO. The in vivo performance of plasmonic nanobubbles as cell theranostic agents in zebrafish hosting prostate cancer xenografts. Biomaterials 31:7567-7574 (2010).
33. Lukianova-Hleb E, Bezek S, Szigeti R, Khodarev A, Kelley T, Hurrell A, Berba M, Kumar N, D'Alessandro U, Lapotko D. Transdermal Diagnosis of Malaria Using Vapor Nanobubbles. Emerg Infect Dis. 22:344 (2016).
34. Hleb EY, Lapotko DO. Photothermal properties of gold nanoparticles under exposure to high optical energies. Nanotechnology 19:355702 (2008).
35. Lukianova-Hleb EY, Anderson LJE, Lee S, Hafner JH, Lapotko DO. Hot plasmonic interactions: A new look at the photothermal efficacy of gold nanoparticles. Phys Chem Chem Phys 12:12237-12244 (2010).
36. Lukianova-Hleb EY, Volkov AN, Lapotko DO. Laser pulse duration is critical for the generation of plasmonic nanobubbles. Langmuir 30:7425-7434 (2014).
37. Yameen B, Choi WI, Vilos C, Swami A, Shi J, Farokhzad OC. Insight into nanoparticle cellular uptake and intracellular targeting. J Control Release 190:485-499 (2014).
38. Hoshyar N. Gray S, Han H, Bao G. The effect of nanoparticle size on in vivo pharmacokinetics and cellular interaction. Nanomedicine (Lond) 11(6):673-692 (2016).
39. Lammel T, Mackevica A, Johansson BR, et al. Endocytosis, intracellular fate, accumulation, and agglomeration of titanium dioxide (TiO₂) nanoparticles in the rainbow trout liver cell line RTL-W1. Environ Sci Pollut Res 26:15354-15372 (2019).
40. Guler U, Shalaev VM, Boltasseva A. Nanoparticle plasmonics: going practical with transition metal nitrides. Materials Today 18:227-237 (2015).
41. Guler U, Suslov S, Kildishev AV. Boltasseva A, Shalaev VM. Colloidal plasmonic titanium nitride nanoparticles: properties and applications. Nanophotonics 4:269-276 (2015).
42. Sabzeghabae AN, Berrospe-Rodriguez C, Mangolini L, Aguilar G. Laser-induced cavitation in plasmonic nanoparticle solutions: A comparative study between gold and titanium nitride. J Biomed Mater Res A 109(12):2483-2492 (2021).
43. He W, Ai K, Jiang C, Li Y, Song X, Lu L. Plasmonic titanium nitride nanoparticles for in vivo photoacoustic tomography imaging and photothermal cancer therapy. Biomaterials 132:37-47 (2017).
44. Bubnov AA, Syui AV, Popov AA, et al. Comparative study of photothermal conversion efficiency in aqueous suspensions of silicon and titanium nitride nanoparticles for biomedical applications. Phys Atom Nuclei 86, 2743-2747 (2023).
45. Schramke KS, Qin Y, Held JT, Mkhoyan KA, Kortshagen UR. Nonthermal plasma synthesis of titanium nitride nanocrystals with plasmon resonances at near-infrared wavelengths relevant to photothermal therapy. ACS Appl Nano Mater 1:2869-2876 (2018).
46. Wang C, Dai C, Hu Z. Li H, Yu L. Lin H, Bai J, Chen Y. Photonic cancer nanomedicine using the near infrared-Il biowindow enabled by biocompatible titanium nitride nanoplatforms. Nanoscale Horiz 4(2): 415-425 (2019).
47. Zelepukin I, Popov A, Shipunova V, Tikhonowski G, Mirkasymov A, et al. Laser-synthesized TiN nanoparticles for biomedical applications: Evaluation of safety, biodistribution and pharmacokinetics. Materials Science and Engineering: C 120:111717 (2021).
48. Popov AA, Tselikov G. Dumas N, et al. Laser-synthesized TiN nanoparticles as promising plasmonic alternative for biomedical applications. Sci Rep 9:1194 (2019).
49. Guler U. Ndukaife JC, Naik GV, Nnanna AG, Kildishev AV, Shalaev VM, Boltasseva A. Local heating with lithographically fabricated plasmonic titanium nitride nanoparticles. Nano Lett 13:6078-6083 (2013).
50. Krekeler T, Rout SS, Krishnamurthy GV, et al. Unprecedented thermal stability of plasmonic titanium nitride films up to 1400° C. Adv Opt Mater 9:2100323 (2021).

1. A method for diagnosing a tissue in a patient comprising: (a) administering metal nanoparticles to a patient; (b) navigating a probe to a target tissue; (c) generating plasmonic nanobubbles with a pump laser pulse delivered through the probe; (d) detecting plasmonic nanobubbles optically in vivo; and (e) diagnosing the target tissue through analysis of the detected optical signal in response to a pump laser pulse.

2. A method of claim 1, wherein the nanoparticles comprise particles capable of developing transient non-stationary plasmon resonance properties during their exposure to a pump laser pulse, such properties absent under exposure of the particles to continuous pump laser beam or a pulsed laser beam with suboptimal duration and intensity of a pump laser pulse.

3. A method of claim 1, wherein the nanoparticles comprise particles conjugated with cancer-specific molecules.

4. A method of claim 1, wherein the nanoparticles comprise two or more types of nanoparticles, wherein each type of nanoparticles comprises different plasmonic properties stationary or transient non-stationary, capable of generating plasmonic nanobubbles while exposed to two or more simultaneous pump laser pulses having different wavelengths that match plasmonic properties of said nanoparticles.

5. A method of claim 1, wherein the nanoparticles comprise metal nitride ceramic nanoparticles of the size in the range from 10 nm to 100 nm, said nanoparticles are exposed to laser pulses of the wavelength above 800 nm and duration above 200 ps at the laser fluence above the threshold of plasmonic nanobubble generation.

6. A method for optical detection of plasmonic nanobubbles in tissue [in vivo] comprising:
(a) illuminating a pump laser-exposed tissue with a probe laser light at a time when a pump laser pulse arrives into the tissue;
(b) collecting the probe laser light scattered by the pump-laser exposed tissue to a photodetector capable of measuring the relative temporal changes in the intensity and power of the collected probe laser light;
(c) detecting a relative change in the intensity of a probe laser light scattered by plasmonic nanobubble;
(d) identifying one or more output signal component specific for a plasmonic nanobubble.

7. A method of claim 6, wherein the one or more output signal component comprises bell-shaped signal components with a peak, negative or positive, relative to the signal baseline.

8. A method of claim 6 for detecting target cells with plasmonic nanobubbles, comprising:
(a) exposing the cells to one or more pump laser pulses at specific wavelength, duration and fluence in the range from 20 mJ/cm2 to 150 mJ/cm2;
(b) exposing the same cells to a probe laser light at the time it receives a pump pulse;
(c) collecting and analyzing a probe laser light as an optical signal;
(d) deriving quantitative parameters from the signal within the time interval from 5 ns to 2 us after the exposure of the tissue to a pump laser pulse;
(e) comparing such quantitative parameters against pre-determined diagnostic thresholds for a target cell type;
(f) determining the presence of the target cell type, wherein the target cell type is present if one or more parameters of the signal of the collected probe laser light match a diagnostic threshold.

9. A method of claim 6, further comprising running an algorithm that compares the quantitative parameters of the probe laser light-detected signals (detected in response to pump laser pulses) to pre-determined thresholds, wherein the comparison concludes whether the cells are disease-positive or-negative (without a human decision being involved), wherein in the case of disease-positive conclusion, the method further comprises the generation of additional pump laser pulses of the increased fluence to the same location, while the probe remains in contact with tissue, with the fluence increased to the level in the range from 100 mJ/cm2 to 250 mJ/cm2.

10. A method of claim 6 for intraoperative automated detection of residual cancer cells in a surgical cavity comprising:
(a) a probe brought in optical contact with the cavity tissue at specific location of a surgical cavity;
(b) applying a pump laser pulse and a probe laser light;
(c) collecting and analyzing the probe laser light;
(d) automatically determining cancer status of a cavity tissue in contact with the probe;
(e) producing the diagnostic data, including the cancer status and the location of the probe.

11. A method of claim 6 for eradication of cancer cells in a target tissue comprising:
(a) detecting cancer cells in a target tissue using the method of claim 6;
(b) exposing the target tissue with a pump laser pulse while the probe remains in optical contact with the diagnosed tissue;
(c) applying one to twenty pump laser pulses at specific wavelength, duration, and fluence in the range from 70 mJ/cm2 to 250 mJ/cm2;
(d) delivering pump laser pulses to the same tissue location through the probe;
(e) monitoring the therapeutic effect of each pump laser pulse through optical detection of plasmonic nanobubbles, with quantitative parameters derived for each optical signal;
(f) comparing the signal parameters against pre-determined therapeutic thresholds; and
(g) adjusting the fluence of next pump laser pulse if the signal parameters did not match the therapeutic thresholds during the previous laser pulse.

12. A device for optical detection of plasmonic nanobubbles in tissue [in vivo] comprising:
(a) a probe laser beam;
(b) a pump laser beam;
(c) an interface optical element, wherein the interface optical element collects probe laser light scattered or reflected by plasmonic nanobubbles generated in tissue volume exposed to laser beams;
(d) a flexible optical guide capable of delivering the collected probe laser light to one or more remote photodetectors, wherein the one or more remote photodetectors can generate an electrical output signal specific to a plasmonic nanobubble.

13. A device of claim 12 for optical generation of plasmonic nanobubbles in tissue [in vivo] comprising:
(a) a probe with a flexible optical guide connected to a distal optical probe, capable of delivering a pump laser pulse, wherein the flexible optical guide is configured to deliver pump and probe laser beams from one or more sources to a probe in contact with tissue without distorting spectral, temporal and energy properties of the laser beams;
(b) an interface optical element in the probe capable of providing an exposure of the tissue volume with a pump and probe laser beams;
(c) a tip of the flexible optical guide capable of being positioned at a distance from about 5 um to about 2 mm from a back surface of the interface optical element, wherein the combination of the distance between the tip and the back surface of the interface optical element, and thickness and refractive properties of the interface optical element forms a diameter of the pump laser beam in the range from 20 to 200 um in tissue in the tissue depth range from about 0 um to about 400 um from the probe surface, and wherein the direction of the pump laser beam in the tissue along with optical axis of the flexible optical guide in the probe is capable of generating plasmonic nanobubbles in front of the probe.

14. A device of claim 12, wherein the probe laser beam and the pump laser beam are capable of being co-delivered into tissue such that both beams overlap or coincide in the tissue.

15. The device of claim 12, wherein the probe laser beam comprises a diameter, wherein the diameter is limited to minimize the background of the light scattered by the tissue.

16. The device of claim 12, further comprising an optical element in the probe capable of delivering the pump and probe laser beams from the fiber guide to the tissue without focusing them, i.e. maintaining the desired diameter D in the tissue near the probe, and to collect and collimate the probe laser light scattered by a plasmonic nanobubble generated in the tissue near the probe.

17. A device of claim 12, wherein the probe laser beam is capable of being internally reflected from or scattered by the optical interface surface between the tissue and the probe such that a scattering of the probe laser light changes when a plasmonic nanobubble is generated in the tissue close to the interface surface, and wherein said changes in the scattering of the probe laser light are capable of being optically detected as a signal associated with the plasmonic nanobubble.

18. A device of claim 12, further comprising a photodetector at the proximal end of the probe that has a single photosensitive element capable of converting a probe laser light intensity, phase, polarization, duration or wavelength into an electrical signal of a time-amplitude or space-amplitude type.

19. A device of claim 12, wherein the probe is routed through standard minimally invasive clinical tools, flexible endoscopes (or similar endo-tools like bronchoscope and endomicroscope or else), biopsy needles or catheters.

20. A device of claim 12, further comprising a compact optical probe of the diameter not to exceed 2 mm, wherein the compact optical probe is capable of optically generating and detecting plasmonic nanobubbles in tissue near the tissue-probe interface, wherein the compact optical probe comprises a free-space optical guide capable of transmitting pump and probe laser beams through a rigid guide from laser sources to the probe, and from the probe to the photodetector(s) and signal hardware and software.
